(19) United States
(12) Patent Application Publication
(10) Pub. No.: US 2024/0024575 A1
(43) Pub. Date: Jan. 25, 2024

(54) METHOD FOR REDUCING A BOLUS, FORECAST METHOD, SAFETY DEVICE, AND MEDICAL PUMP

(71) Applicant: B. Braun Melsungen AG, Melsungen (DE)

(72) Inventors: Dennis Obermann, Willingshausen (DE); Thomas Wiegand, Homberg (Efze) (DE); Hans-Christian Moritzen, Kassel (DE)

(21) Appl. No.: 18/354,949

(22) Filed: Jul. 19, 2023

(30) Foreign Application Priority Data
Jul. 20, 2022 (DE) 10 2022 118 179.0

(51) Int. Cl. A61M 5/168

(52) U.S. Cl. CPC A61M 5/16854; A61M 2005/14208

(57) ABSTRACT

A method for reducing a bolus in a fluid guide unit, a forecast method, a safety device and a medical pump. The method for reducing a bolus includes detecting a pressure course in the fluid guide unit by a sensor unit, storing the pressure course in a memory unit; determining an occlusion probability by a control unit based on the pressure course, detecting a volume delivered by the pump for an operating range with high occlusion probability, which can be based on a detected number of motor steps of a pump motor or of a detected rotation angle of the pump motor, detecting an occurrence of an occlusion at a detection time by a detection unit when exceeding a pressure limit value, and pumping back the delivered volume for the operating range with high occlusion probability, in particular by retracting the detected motor steps or the detected rotation angle.

### CROSS-REFERENCE TO RELATED APPLICATION(S)

This application claims priority under 35 U.S.C. § 119 to German Application No. 10 2022 118 179.0, filed on Jul. 20, 2022, the content of which is incorporated by reference herein in its entirety.

### FIELD

The present disclosure relates to a method for reducing a bolus in a fluid guide unit, in particular in an outlet line, of a medical pump, preferably of an infusion pump. In addition, the present disclosure relates to a forecast method for determining an occlusion probability, a safety device for reducing a bolus and a medical pump, in particular an infusion pump.

### BACKGROUND

Occlusions or blockades/obstructions can occur in medical pumps, in particular infusion pumps. Occlusions can occur, for example, due to kinking or twisting of elastic hoses, for example unintentionally due to corresponding handling. In particular, an occlusion in a fluid guide unit, in particular an outlet line, of the pump, i.e. a section in which the fluid is delivered to the patient, is critical. Due to the occlusion, infusion fluid in particular can collect in the elastic outlet line, which is configured as a disposable article. The medical fluid accumulated due to the occlusion represents a bolus in this case. When the occlusion is removed, i.e. the obstacle to the fluid is removed, the accumulated volume of infusion fluid may inconveniently or unintentionally be administered to the patient all at once. This intensive and rapid administration can be harmful to the patient, in particular with critical drugs, and in extreme cases may be even lethal. Furthermore, the pressure in the fluid guide unit may rise above a desired safety level due to the occlusion, which can also lead to damage to the patient or the fluid guide unit.

It is therefore known to equip medical pumps with occlusion detection or a unit for occlusion detection, respectively. Occlusion detection is based on pressure measurements from pressure sensors. In the event of an occlusion in the outlet line of the pump, the pressure in the outlet line increases accordingly. The pressure sensor(s) detect the pressure increase and switch off the pump as quickly as possible. In order to prevent the accumulated infusion fluid from being administered to the patient unintentionally, known pumps with occlusion detection have a function for so-called bolus reduction/reduction of a bolus. This involves pumping out a fixed/determined volume calculated on the basis of a pressure limit value, recently even together with a characteristic value/parameter of the disposable article. This is based on the assumption that the structure of the outlet line, i.e. the composition of the various disposable articles, is always the same for different applications and that the occlusion is always located at the same place in the outlet line. In a sense, a single fixed value is set for the disposable article and the pump and this fixed, standardized value is used in all situations of an occlusion.

However, experience in practice has shown that such assumptions do not hold true. Indeed, the bolus volume depends not only on a pressure in the line, but also on other factors such as the material of the disposable articles, the geometry of the disposable articles, the structure of the clinical set-up, the ambient temperature, and in particular also the site or location of the occlusion. It is therefore not sufficient to simply pump back a fixed volume of conveyed fluid.

Solutions are known from the prior art that estimate the time of an occurrence of the occlusion and, based on this estimated time, provide an estimate of what the volume of the bolus might be. The estimated volume is then pumped back by the pump.

A method for analyzing pressure changes in an infusion device is known from EP 1 136 089 B1. The infusion device has several modules. When a pressure change is detected in a line, an analysis is performed to determine an involvement of other modules in the pressure change. A pressure course is retrospectively analyzed in order to determine the time of the occurrence of an occlusion. In particular, pressure changes indicate the occurrence of the occlusion. A time span between an occurrence time of the occlusion and a detection time of the occlusion is determined. After detection of the occlusion, infusion fluid is pumped back for this time span. This reduces the bolus.

US 2021/0 330 881 A1 and WO 2021/042 126 A1 both disclose a system for reducing a bolus after detection of an occlusion. Fluid is pumped back after detection of the occlusion until a predetermined, safe pressure level is reached, from which it is assumed that the occlusion has been eliminated. However, this reduction of the bolus is very inaccurate.

However, all known solutions have the common disadvantage that the estimation of the bolus volume is currently very inaccurate. Therefore, in the worst case, the pump may pump back the wrong volume, so that either too much fluid is pumped out or a bolus remains in the fluid guide unit, such as the outlet line.

### SUMMARY

Thus, the objects of the present disclosure are to overcome or at least reduce the disadvantages of the prior art and, in particular, to provide a method, a forecast method, a safety device and a medical pump with which a volume of an (unwanted) bolus can be safely and reliably reduced or reset when an occlusion occurs. A partial object can be seen in detecting in particular a volume of a fluid that has been delivered after an occlusion has occurred and pumping back this volume accordingly.

The present disclosure relates to a method for reducing an (unwanted) bolus or accumulation of a fluid in a fluid guide unit (downstream of the pump), in particular an outlet line, of a medical pump, preferably an infusion pump. The method according to the disclosure comprises the following steps. A sensor unit detects, in particular continuously or repeatedly at discrete time intervals, a pressure course in the fluid guide unit, in particular in the outlet line. A memory unit preferably stores the detected pressure course in order to be able to analyze it as a whole at a later time and to determine a change over time. A control unit determines an occlusion probability, i.e. a probability between 0% and 100% that an occlusion will occur, based on the detected pressure course. The pressure course detected over time can therefore be used to determine a probability of occlusion using a corresponding analysis. For an operating range of the pump with a high occlusion probability (i.e. an occlusion probability above a predefined limit value that separates an operating range of a low occlusion probability from a high occlusion probability), the control unit detects (from this time of the change to the operating range of the high occlusion probability) a volume delivered by the pump. In particular, a number of performed motor steps of a pump motor is detected or a rotation angle of the pump motor or a number of revolutions of the pump or pump motor, respectively. If the medical pump delivers fluid using peristalsis, a peristalsis movement can also be detected. A detection unit detects/determines an occlusion occurring at a detection time (i.e. after an occurrence time) when an alarm condition is exceeded, in particular a pressure limit value as alarm condition. The volume pumped by the pump for the determined operating range with high occlusion probability is then pumped back by the pump accordingly. In particular, the detected motor steps or the detected rotation angle are retracted by the pump motor.

The operating range with high occlusion probability is determined based on the detected pressure course. When the pump is operated in the range of high occlusion probability (or changes into this range), the motor steps of the pump motor or the number of revolutions or something similar are detected (from the start of the operating range of high occlusion probability). The number of revolutions or motor steps or rotation angles are preferably detected by the control unit and stored in the memory unit (e.g. the number of revolutions recorded over time). If the detection unit detects an occlusion at a detection time, an alarm or the like can preferably be issued and the pump is stopped. The detected (or stored) motor steps or revolutions correspond with a certain probability to the steps performed since the occlusion occurred (i.e. since the occurrence time of the occlusion) but had not yet been detected (i.e. before the detection time). Thus, the motor steps or the rotation angle between the occurrence and the detection of the occlusion can be determined or at least estimated without knowing the exact time of the occurrence of the occlusion. A time span between the occurrence and the detection of the occlusion is a time span between the occurrence time of the occlusion and the detection time of the occlusion. The detected motor steps or number of revolutions or rotation angles are retracted by the pump motor after the detection of the occlusion at the detection time, so that the unwanted bolus is safely removed from the fluid guide unit, in particular the outlet line of the pump.

The occlusion probability is the probability (between 0% and 100%) that an occlusion occurs and is determined by the control unit based on the detected pressure course. A high occlusion probability can be present if the probability of an occlusion occurring, which is output by the control unit, is higher than a limit value, for example 50%, preferably higher than 70%. In particular, the predefined limit value may be variably adjustable before the start of a treatment. However, other limit values for a change of the operating mode from the low to the high occlusion probability are also conceivable, as long as they have a positive value, i.e. also about 20%. If now an occlusion probability is estimated and this exceeds a limit value or an alarm condition, then in a sense an occurrence of an occlusion can be determined directly. Thus, in contrast to the prior art, it is no longer necessary to retrospectively go back from a relative detection time, but rather two times are determined, so to speak, an (estimated) occurrence time (i.e., an operating range of high occlusion probability) and a detection time, and a pump volume is already determined individually for this time range. In this way, a bolus volume can be determined and pumped back even more precisely.

For example, the control unit outputs a control signal that causes the pump motor to retract the detected motor steps or the detected rotation angle or the detected number of revolutions.

The alarm condition or the limit value above which an occlusion is detected may, for example, be a (predetermined) pressure limit value. The pressure sensors are already present in the fluid line. Furthermore, exceeding a predefined occlusion probability can preferably also indicate the occurrence of occlusion. In particular, the occlusion probability for detection has to be even higher than the high occlusion probability. The occurrence of an occlusion can preferably also be detected by exceeding a certain number of motor steps and/or a certain time, preferably when the pump has been pumping for a certain time without resulting in a volume flow.

In summary, the essence of the disclosure is to detect the (operating) mode with high occlusion probability (and to predict an occurrence of an occlusion, so to speak), to detect the pump volume, in particular the motor steps of the pump motor, in or respectively from this mode (of high occlusion probability) and, upon (actual) detection of the occlusion, to retract the detected motor steps between the occurrence time and the detection time of the occlusion and thus to pump back the volume delivered by the pump between the occurrence and the detection of the occlusion.

The method according to the disclosure allows the bolus volume to be estimated very accurately. This enables the pump to pump the bolus volume very precisely. The signal that changes the occlusion probability from small/low to high starts the detection of the pump volume, in particular the motor steps. The change of the operating mode from low to high occlusion probability corresponds with a probability (the occlusion probability) to the occurrence time of the occlusion. From this time onwards, the pumped volume is detected, in particular the motor steps performed. Thus, the volume delivered or, respectively, the motor steps since the (estimated) occurrence time of the occlusion are detected without having to determine the exact occurrence time of the occlusion. The occlusion probability is output in real time (i.e. current) based on the detected pressure course. By detecting the volume delivered between the (estimated) occurrence time of the occlusion and the detection time of the occlusion, in particular by detecting the motor steps performed or the rotation angle of the pump motor, precisely this detected volume can be pumped back or the motor steps or the rotation angle can be retracted. As a result, the bolus is completely removed or is at least greatly minimized. Dangerous administration of a large quantity of medication to the patient at once is thus completely prevented or is at least much less likely.

The method according to the disclosure makes the determination of the bolus volume independent of the clinical set-up, the material characteristics of the disposable articles, in particular the geometry and material, the environmental conditions such as the temperature and the place of occlusion. This can reduce the (unwanted) bolus and increase patient safety.

The object of the present disclosure is further solved by a forecast method for determining the occlusion probability. The forecast method comprises the following steps. The sensor unit detects (historical) pressure courses. Associated (historical) occurrences of occlusion events are also detected. The detected pressure courses and the detected occurrences of occlusion events are combined to a training data set. The system for machine learning is trained with the training data set. In particular, the detected (historical) pressure courses are the input values and the occurrences of (historical) occlusion events are the output values of the system for machine learning. During a pump operation, the (current) pressure course is detected and input to the trained system for machine learning in real time. The trained system for machine learning (as a forecast method) outputs an estimate/prediction of the occlusion probability in real time, based on the input current pressure course.

The forecast method can essentially be divided into two different phases. In a training phase, the system for machine learning is trained with historical data. The historical data was detected in the past during test runs or already completed pumping procedures or infusions, respectively. It is important that a detected pressure course is always associated with information on whether or not an occlusion has occurred. If the information with the detected pressure course and the information about the occlusion occurrence are linked to an associated data set, the system for machine learning can be trained with this data set. As a result, the system for machine learning can associate the relationship between a particular pressure course and the occurrence of the occlusion and determine, for example, a probability that this pressure course is associated with an occlusion, for example, via a correlation of a new pressure course with one or more pressure courses and within a tolerance range. For example, a deviation of the pressure course can be used to indicate a probability. Thus, the system for machine learning learns an occlusion probability for the specific pressure course. The trained system for machine learning is the starting point for a second phase, the forecast phase. In the forecast phase, a currently detected pressure course is input into the trained system for machine learning. The trained system for machine learning outputs the occlusion probability for this pressure course in real time or, respectively, a statement/estimate/prediction as to whether occlusion will occur or not. By outputting the occlusion probability by the system for machine learning, the operating mode of the medical pump can be changed from the operating mode with high occlusion probability to the operating mode with low occlusion probability or vice versa. The change of the operation mode from low occlusion probability to high occlusion probability is in particular an estimation of the occurrence time of the occlusion.

In particular, the output of the system for machine learning may be a percentage indicating the occlusion probability. For example, a threshold value may be specified wherein the occlusion probability is a high occlusion probability if the threshold value is exceeded. For example, this threshold value is at 50%. The system for machine learning may also output a binary value, wherein a ‘one’ indicates a high occlusion probability and a ‘zero’ indicates a low occlusion probability, or vice versa.

The object of the present disclosure is further solved by a safety device/protection device/safety unit/safety module for reducing the (unwanted) bolus or the unwanted accumulation of fluid in the fluid guide unit, in particular an outlet line, of a medical pump, preferably an infusion pump. The (safety) device comprises a sensor unit for detecting a pressure course in the fluid guide unit, in particular a memory unit for storing the detected pressure course, and a detection unit for detecting an occurrence of an occlusion. Furthermore, the device according to the disclosure comprises a control unit for determining the occlusion probability and for detecting a volume delivered by the pump for an operating range with high occlusion probability, in particular a number of motor steps of a pump motor or a rotation angle of the pump motor.

The control unit also sends the (control) signal for retracting the determined motor steps or the number of revolutions or the rotation angle to the pump motor. In this way, quite analogously to the method of the present disclosure, the (estimated) delivered volume is determined individually and is returned accordingly in order to completely break down the bolus.

According to an optional aspect of the present disclosure, the following steps are performed to determine the occlusion probability. Preferably, a (trained) system for machine learning is created with the detected pressure course as input value and the occlusion probability as output value. The pressure course detected by the sensor unit can be input to the (previously trained) system for machine learning in real time. The system for machine learning then outputs the occlusion probability based on the detected and input pressure course. The system for machine learning can thus establish a relationship between the pressure course and the occlusion probability. Based on this correlation or a knowledge of a correlation between the pressure course and the occlusion probability, respectively, the system for machine learning can output the occlusion probability depending on the detected pressure course. The output of the occlusion probability is preferably done in real time. By outputting the occlusion probability, the change between the operating mode of the pump with the low occlusion probability and the operating mode of the pump with the high occlusion probability can be determined. This allows the detection of the delivered volume, in particular the motor step or the rotation angle, to be started. With the change between the operating mode with low occlusion probability and the operating mode with high occlusion probability, an estimate of the occurrence or the occurrence time of the occlusion is preferably provided.

The system for machine learning is preferably an artificial neural network. Particularly preferred, neural networks may be used that have been developed for processing time-dependent data sets, such as recurrent neural networks or LSTM networks (long short-term memory).

Preferably, the following steps are performed to create the system for machine learning. The sensor unit detects the pressure course. In particular (historical) pressure courses are detected. Occurrences of occlusion events can be detected (by the control unit). The detected pressure courses and the detected occurrences of occlusion events are combined to a training data set. The system for machine learning can be trained with the training data set. In particular, the detected (historical) pressure courses are used as input values and the occurrences of (historical) occlusion events as output values of the system for machine learning.

The training data set can be created from (historically detected) pressure courses of the pump and (historically detected) occlusion events/occlusion occurrences. Preferably, the pressure courses are the input/feed and the occlusion events are the output/readout of the system for machine learning. Through training, the system for machine learning learns the relationship between the pressure courses and the occurrences of the occlusions. This allows the system for machine learning to learn the occlusion probability as a function of the pressure course. When the pressure course in the fluid guide unit is detected during operation of the medical pump and is input to the system for machine learning, the trained system for machine learning can output the occlusion probability for the pressure course in real time. The control unit of the pump can thus distinguish between a mode with a low and a mode with a high occlusion probability. Thus, the trained system for machine learning is the basis for the targeted detection of the motor steps performed or the rotation angle.

According to a further optional aspect of the present disclosure, the following steps are performed to detect the volume delivered by the pump by the control unit. Preferably, a time of the occurrence of the occlusion is determined. The control unit may determine a time span between the occurrence time of the occlusion and the detection time of the occlusion. The volume that was delivered between the occurrence time of the occlusion and the detection time of the occlusion is preferably determined from the determined time span.

The control unit can preferably determine the time of the occurrence of the bolus by retrospectively observing the detected pressure course after the detection of the occlusion. From the time span between the occurrence time of the occlusion and the detection time of the occlusion, the control unit can calculate the volume that was pumped between the occurrence of the occlusion and the detection of the occlusion with knowledge of a pump delivery rate. This volume can then be pumped back through the pump. This determination of the pumped/delivered volume enables the pump to reduce the bolus in a targeted manner.

In particular, the control unit can be adapted to determine the pumped volume between the occurrence time and the detection time by the number of motor steps or the rotation angle, for example by recording the pumped volume and storing it in a memory unit, and then pumping back this pumped volume accordingly.

Preferably, the following additional steps are performed by the pump in order to pump back the volume delivered by the pump. The pressure in the fluid guide unit, in particular the outlet line, is reduced, for example, by pumping it back with the pump. In parallel/simultaneously, the pressure course is detected by the sensor unit. When the pressure in the fluid guide unit reaches a pressure level, at which the pressure is constant with further pumping back, pumping back is stopped or, respectively, the pump motor is stopped to safely stop pumping back and prevent overpumping. The presence of the bolus can mean an increased pressure level in the fluid guide unit. When the accumulated fluid is pumped out, the pressure in the fluid guide unit is reduced. Once the bolus is pumped out, further pumping will not result in a pressure reduction. Therefore, if pumping occurs and the pressure in the fluid guide unit does not decrease despite pumping, it can be assumed that the bolus has been reduced or pumped down, respectively. For example, this may be due to elastic expansion of the tubing, which has, however, irreversibly expanded to a degree and thus (without overpressure) has a larger volume than before. The pump motor is then stopped. By pumping with parallel pressure detection, the bolus can be pumped out in a targeted manner. This preferably provides a (redundant) protection that prevents that too much fluid is pumped out.

Preferably, the control unit determines the time of the occurrence of the occlusion by deriving the detected pressure course twice and determining the zeros of the second derivative of the detected pressure course and selecting the zero with a minimum value from these zeros. The detected pressure is time-dependent and results in a curve in a pressure-time diagram or in a pressure-volume diagram, wherein the volume is the volume delivered by the pump. The occurrence of the occlusion changes the pressure course. Thus, by looking at the extremes and/or the inflection points of the pressure course, the points where the pressure changes can be determined.

Preferably, the control unit determines the time of the occurrence of the occlusion by simply deriving the detected pressure course and determining a change in the slope of the detected pressure course. In particular, the control unit determines a minimum value at which the slope increases. The pressure course changes as a result of the occlusion. The pressure increases as a result of the occlusion. The control unit determines the time from which the pressure increases. This time can represent the time of the occlusion occurrence.

Preferably, the control unit determines the time of occurrence of the occlusion by determining inflection points of the detected pressure course, wherein a smallest inflection point (an inflection point to a smallest pressure) represents the determined occurrence time of the occlusion.

The object of the present disclosure is further solved by a medical pump comprising a control unit configured and prepared to perform a method according to the preceding aspects.

In particular, the medical pump, especially an infusion pump, may comprise a safety device for reducing a bolus according to the present disclosure.

Any disclosure relating to the method according to the present disclosure also applies to the safety device as well as the medical pump, as well as any disclosure relating to the safety device and the medical pump of the present disclosure also applies to the method of the present disclosure. In particular, the control unit of the safety device or of the medical pump, respectively, may be adapted to perform the method steps of the method.

### BRIEF DESCRIPTION OF THE DRAWINGS

The disclosure is explained in more detail below with reference to preferred embodiments with the aid of figures.

FIG. 1 shows a perspective view of a medical pump according to a preferred embodiment.

FIG. 2 shows a schematic view of a safety device according to a preferred embodiment.

FIG. 3 shows a flow diagram of a method according to the disclosure according to a preferred embodiment for reducing a bolus.

FIG. 4 shows a flowchart of a forecast method according to a preferred embodiment of the present disclosure.

FIG. 5 shows a schematic representation of a system for machine learning.

FIG. 6 shows a diagram illustrating a mode of operation of the present disclosure showing a relationship between a delivered volume of the medical pump and a pressure.

The Figures are schematic in nature and are intended only to aid understanding of the disclosure. Identical elements are provided with the same reference signs. The features of the various embodiments may be interchanged.

### DETAILED DESCRIPTION

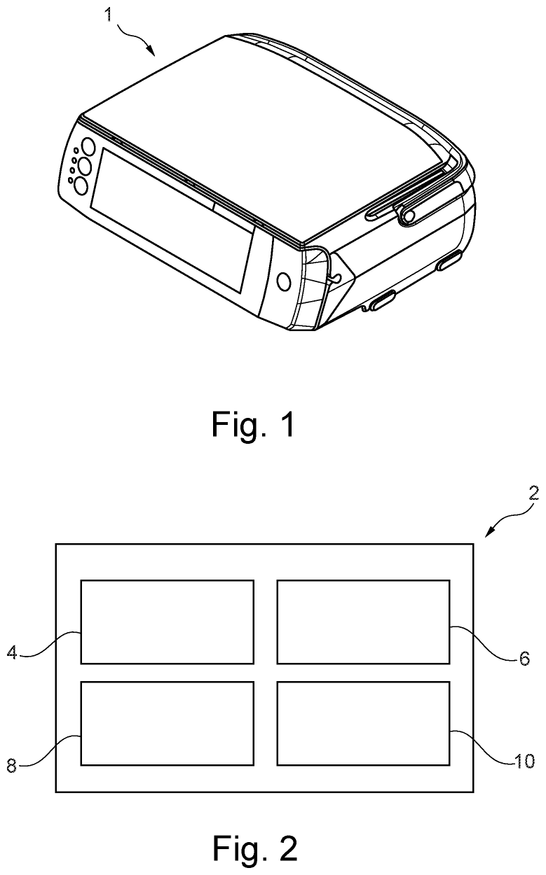

FIG. 1 shows a perspective view of an infusion pump 1. The infusion pump 1 has a fluid guide unit/outlet line (not shown) that leads to a patient (not shown). This outlet line is inserted into a front of the infusion pump 1 and is actuated accordingly by a pump. The outlet line is configured as a disposable article made of an elastic material and is, for example, a hose system. A medical fluid, in particular an infusion fluid, in which a medication for the patient is contained, is conveyed/pumped through the outlet line. Should a blockade/occlusion occur in the outlet line, an (unwanted) accumulation of the fluid, the so-called bolus, can accumulate in the elastic outlet line. When the occlusion is released, this could cause the bolus to be delivered to the patient all at once. This is to be avoided, especially with critical drugs. Therefore, the infusion pump 1 of the present embodiment has a safety device 2 for reducing the bolus in the outlet line or, respectively, a method for reducing the bolus according to the disclosure can be carried out on the infusion pump 1, as explained below for FIGS. 2 to 4.

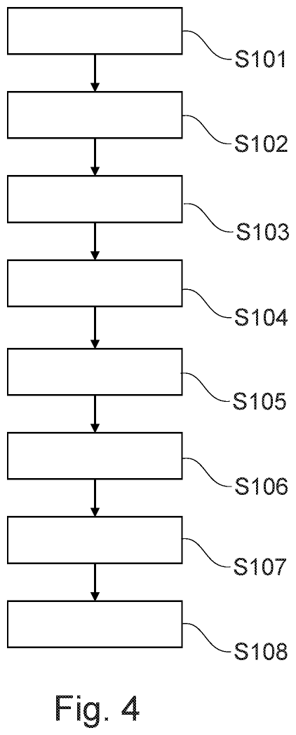

FIG. 2 shows a schematic representation of a configuration of such a safety device 2, which is used in the infusion pump 1. The safety device 2 has a sensor unit 4, a memory unit 6, a control unit 8 and a detection unit 10. The sensor unit 4 detects a pressure course in the outlet line. The memory unit 6 stores the pressure course detected by the sensor unit 4. The control unit 8 determines an occlusion probability, in particular according to a forecast method for determining the occlusion probability, which is explained in detail below in connection with FIG. 4, and detects a volume delivered by the pump 1 for an operating range with (or respectively time-wise from) a high occlusion probability. This also determines an estimated occurrence time of the occlusion. In particular, the control unit 8 detects a number of motor steps of a pump motor (not shown) or a rotation angle of the pump motor. The detection unit 10 detects an occurrence of an occlusion at a detection time in the outlet line when the detected pressure exceeds a predefined pressure limit value.

Based on the determination of the executed motor steps, the control unit sends a (control) signal to the pump motor that the executed motor steps are retracted. This removes the bolus unintentionally generated by the blockade individually and safely. Thus, a simple and reliable safety device 2 and infusion pump 1 are provided, which can safely and accurately establish a state before the occlusion occurs.

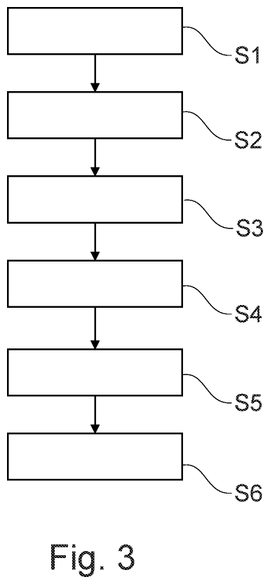

FIG. 3 shows a flowchart of a method according to a preferred embodiment of the present disclosure for reducing a bolus in the outlet line of the infusion pump 1. The infusion pump can in particular be adapted via an appropriately adapted control unit to execute the method.

In a step S1, a pressure course in the outlet line is first detected by the sensor unit 4. A time course is created.

In a step S2, the detected pressure course is stored in the memory unit 6 so that it can be accessed again and the time curve can be analyzed.

In a step S3, the correspondingly adapted control unit 8 determines an occlusion probability based on the detected and stored pressure course.

In a step S4, the control unit 10 detects a volume delivered by the pump for an operating range with high occlusion probability (or from an estimated occurrence time), in particular a number of motor steps of the pump motor or the rotation angle of the pump motor.

In a step S5, the detection unit 8 detects the occurring of the occlusion at a detection time.

In a step S6, the volume delivered by the pump for the detected operating range with high occlusion probability is pumped back by the pump. In particular, the detected motor steps or the detected rotation angle are retracted. This reduces the bolus and breaks it down completely.

Preferably, redundantly, the method may include the step of detecting the pressure by the sensor unit at a current time and stopping pumping back if a predefined pressure level, in particular a pressure level before determining an operating range of high occlusion probability, has been reached to prevent excessive pumping back.

FIG. 4 shows a flowchart of a forecast method according to a preferred embodiment for determining the occlusion probability. The forecast method can be divided into a training phase and a forecast phase.

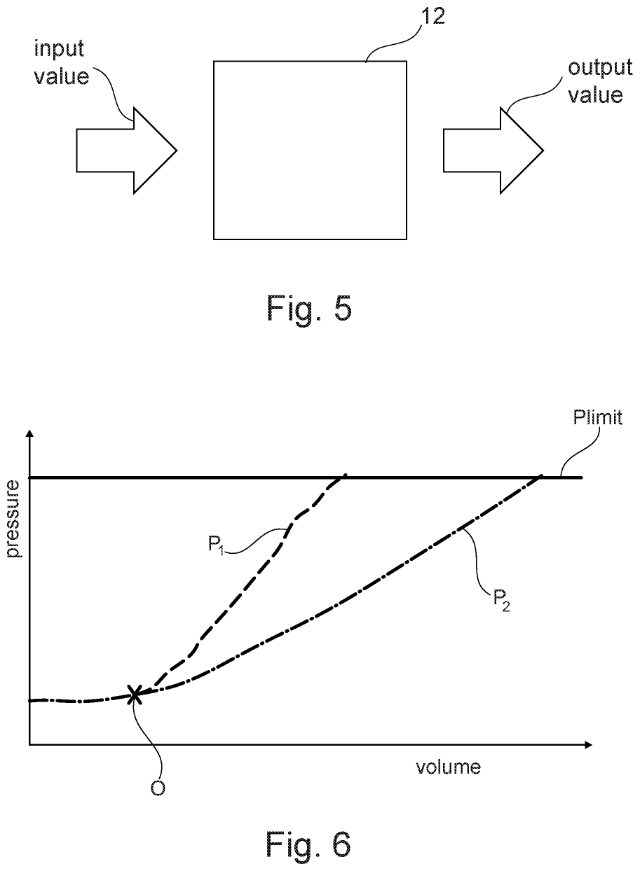

In the training phase, a system for machine learning 12 (shown in FIG. 5) is trained.

In step S101, (historical) pressure courses are detected by the sensor unit 4.

In step S102, occurrences of occlusion events are detected at the detected pressure courses.

In step S103, the detected pressure courses and the detected occurrences of the occlusion events are combined to a (linked) training data set.

In step S104, the system for machine learning is created with a detected pressure course as input value and an occurrence of the occlusion events as output value.

In step S105, the system for machine learning 12 is trained with the training data set. The trained system for machine learning 12 is the final result of the training phase and the starting point of the forecast phase.

In the forecast phase, the system for machine learning 12 is no longer trained with historical data, but the system for machine learning 12 is to make a forecast/estimate given current data.

In step S106, the sensor unit 4 detects a (current) pressure course in real time.

The detected pressure course is input to the trained system for machine learning 12 in step S107.

Based on the entered pressure course, the trained system for machine learning 12 outputs the occlusion probability in step S108.

In this way, a forecast in the form of a percentage probability/estimate of an occlusion can be provided.

FIG. 5 shows a schematic representation of the system for machine learning 12. The system for machine learning 12 is preferably an (artificial) neural network and has an input layer and an output layer. Input values are input into the input layer. In the present case, the input values are a pressure course. An output value is output from the output layer. The output value is the occlusion probability.

The system for machine learning 12 thus maps a relationship between a pressure course as input value and a correlating occlusion probability as output value.

FIG. 6 shows a diagram illustrating the function of the present disclosure, which shows a correlation between the delivered volume of the pump 1 and a pressure in the outlet line. In a first region, the pressure is substantially constant. That is, the pump continuously delivers a volume and the pressure in the outlet line remains correspondingly constant or respectively at the same pressure level, since the delivered fluid can flow away unimpeded. A fluid flow is formed, so to speak. If an occlusion occurs in the outlet line (occurrence time of the occlusion), the pressure rises accordingly when further fluid is pumped into the outlet line by the pump. The pressure continues to rise until a pressure limit value Pₗᵢₘᵢₜ is exceeded. If the pressure limit value Pₗᵢₘᵢₜ (detection time of the detection of the occlusion) is exceeded, an alarm is output and the pump 1 is stopped. The bolus volume depends on the slope of the pressure after the bolus occurs.

If the pressure rises rapidly after occlusion occurs, only a small volume has usually been delivered between occlusion occurring and the pressure limit value being exceeded as an alarm condition (for example, the outlet line is short and does not represent a sluggish, but rather a rapidly reacting system). Accordingly, the bolus has a small volume (curve P1). If, on the other hand, the pressure rises more slowly after the occlusion occurs, more volume is delivered between the occurring of the occlusion (or estimated occurrence time) and the pressure limit value (detection time) being exceeded than if the pressure rises quickly. The bolus volume is therefore greater (curve P2).

From the curve of the pressure course, the occurrence time of the occlusion can be determined by retrospective analysis. The volume that was delivered between the occurrence time of the occlusion and the detection time of the occlusion can thus be determined by retrospective analysis.

In particular, the occurrence time of the occlusion can be determined by the following ways:
Forming the second derivative of the pressure course and zero-point analysis of the second derivative;
Analyzing the slope of the pressure course;
Searching for a smallest inflection point by a search algorithm.

When an occlusion occurs, the slope of the pressure course changes. Therefore, the occurrence time of the occlusion can be determined by analyzing the slope or searching for a point of slope change. This knowledge can be used to determine the occurrence time of the occlusion.

The control unit 10 may be a computer unit, a processor, a motor control unit, a microcontroller or the like. The only prerequisite is that the control unit 10 is prepared and configured to carry out the method or methods according to the disclosure.

What is claimed:

1. A method for reducing a bolus in a fluid guide unit comprising the steps of:
detecting a pressure course in the fluid guide unit by a sensor unit;
determining an occlusion probability by a control unit based on the pressure course;
detecting a volume delivered by a pump for an operating range with high occlusion probability by the control unit;
detecting an actual occurrence of an occlusion at a detection time by a detection unit when an alarm condition is exceeded; and
pumping back the volume delivered by the pump for the operating range with high occlusion probability.

2. The method according to claim 1, further comprising a step of storing the pressure course in a memory unit after the pressure course is detected.

3. The method according to claim 1, wherein the step of detecting the volume delivered by the pump comprises detecting a number of motor steps of a pump motor or of a rotation angle of the pump motor.

4. The method according to claim 3, wherein the step of pumping back the volume delivered by the pump comprises retracting the number of motor steps of the pump motor or the rotation angle of the pump motor.

5. The method according to claim 1, wherein the alarm condition is a pressure limit value.

6. The method according to claim 1, wherein the step of determining the occlusion probability comprises the following steps:
creating a trained system for machine learning with the pressure course as an input value and the occlusion probability as an output value;
inputting the pressure course in real time into the trained system for machine learning; and
outputting the occlusion probability in real time by the trained system for machine learning based on the pressure course.

7. The method according to claim 6, wherein the step of creating the trained system for machine learning comprises the following steps:
detecting the pressure course by the sensor unit;
detecting occurrences of occlusion events;
combining the pressure course and the occurrences of occlusion events into a training data set; and
training the trained system for machine learning with the pressure course as the input value and the occurrences of occlusion events as the output value.

8. The method according to claim 7, wherein the trained system for machine learning is a neural network.

9. The method according to claim 1, wherein the step of detecting the volume delivered by the pump comprises the following steps:
determining an occurrence time of the occlusion;
determining a time span and/or a number of revolutions of the pump between the occurrence time of the occlusion and the detection time of the occlusion by the control unit; and
determining a volume delivered by the pump between the occurrence time of the occlusion and the detection time of the occlusion from the time span and/or the number of revolutions of the pump by the control unit.

10. The method according to claim 9, wherein the occurrence time of the occlusion is a time of a beginning of an operating range with high occlusion probability.

11. The method according to claim 1, wherein the step of pumping back the volume delivered by the pump further comprises:
reducing pressure by pumping back fluid through the pump and simultaneously detecting the pressure course through the sensor unit; and
stopping pumping when the pressure reaches a pressure level at which the pressure is constant to safely stop pumping back and prevent overpumping.

12. A forecast method for determining an occlusion probability comprising the steps of:
detecting pressure courses by a sensor unit;
detecting occurrences of occlusion events at the pressure courses;
combining the pressure courses and the occurrences of occlusion events into a training data set;
creating a system for machine learning having the pressure courses as input values and the occurrences of occlusion events as output values;
training the system for machine learning with the training data set;
detecting a subsequent pressure course in real time by the sensor unit;
inputting the subsequent pressure course into the system; and
outputting an estimate of the occlusion probability in real time based on the subsequent pressure course by the system.

13. A safety device for reducing a bolus in a fluid guide unit of a medical pump, the safety device comprising:
a sensor unit adapted to detect a pressure course in the fluid guide unit;
a detection unit adapted to detect an occurrence of an occlusion; and
a control unit adapted to detect a volume delivered by the medical pump for an operating range with high occlusion probability.

14. The safety device according to claim 13, wherein the control unit is configured to determine an occurrence time of the occlusion by one of the following:
calculating a first derivation of the pressure course and a second derivation of the pressure course, determining zeros of the second derivation of the pressure course, and selecting a zero with a minimum value from the zeros;
deriving the pressure course and determining a change in a slope of the pressure course to a minimum value at which the slope increases; and
determining inflection points of the pressure course, wherein a smallest inflection point represents the occurrence time of the occlusion.

15. The safety device according to claim 13, wherein the control unit is adapted to classify an occlusion probability as high if the occlusion probability exceeds a predefined limit value.

16. The safety device according to claim 15, wherein the safety device is adapted for the predefined limit value to be variably adjustable before a start of a treatment.

17. The safety device according to claim 13, wherein the control unit is adapted to detect a volume delivered by the medical pump by detecting a number of motor steps of a pump motor or a rotation angle of the pump motor.

18. A medical pump comprising:
a control unit configured to perform the method of claim 1; and
a safety device comprising:
a sensor unit adapted to detect a pressure course in the fluid guide unit;
a detection unit adapted to detect an occurrence of an occlusion; and
a control unit adapted to detect a volume delivered by the medical pump for an operating range with high occlusion probability.

19. The medical pump according to claim 18, wherein the fluid guide unit is an outlet line of the medical pump.

20. The medical pump according to claim 18, wherein the medical pump is an infusion pump.
